(19) United States
(12) Patent Application Publication
(10) Pub. No.: US 2022/0154283 A1
(43) Pub. Date: May 19, 2022

(54) MARKER COMPOSITION FOR DIAGNOSING CANCER OR PREDICTING PROGNOSIS ON BASIS OF EXOSOME OVEREXPRESSING TUBA1C PROTEIN

(71) Applicants: KOREA UNIVERSITY RESEARCH AND BUSINESS FOUNDATION, Seoul (KR); KOREA ADVANCED INSTITUTE OF SCIENCE AND TECHNOLOGY, Daejeon (KR)

(72) Inventors: Hyun Koo KIM, Seoul (KR); Sung Hoi HONG, Seoul (KR); Yong PARK, Seoul (KR); Yeon Ho CHOI, Seoul (KR); Byeong Hyeon CHOI, Seoul (KR); Ji Ho PARK, Daejeon (KR)

(21) Appl. No.: 17/442,012

(22) PCT Filed: Apr. 3, 2020
(86) PCT No.: PCT/KR2020/004589

§ 371 Date: Sep. 22, 2021

(30) Foreign Application Priority Data
Apr. 3, 2019 (KR) 10-2019-0039052
Apr. 3, 2020 (KR) 10-2020-0040887

(51) Int. Cl. C12Q 1/6886

(52) U.S. Cl. CPC C12Q 1/6886

(57) ABSTRACT

According to an embodiment of the present disclosure, there is provided a marker composition for diagnosing cancer or predicting prognosis, comprising exosomes overexpressing a Tubulin alpha-1C chain (TUBA1C) protein.

### TECHNICAL FIELD

The following description relates to a marker composition for diagnosing cancer or predicting prognosis, including exosomes overexpressing Tubulin alpha-1C chain (TUBA1C) protein.

### BACKGROUND ART

A tumor is a result of uncontrolled and disordered cell proliferation caused by an excess of abnormal cells. If such a tumor has destructive proliferation, invasion and metastasis, it is classified as a malignant tumor, that is, cancer.

At present, examination means for diagnosing cancer include methods using X-ray imaging, endoscopy, biopsy, etc. However, in spite of the advantage that the examination process is relatively simple, these methods have a problem that the diagnosis success rate is not high, hygiene is poor, and the patient suffers in the course of the examination, thus there is a need for a method for diagnosing cancer to replace these methods.

In order to treat cancer, it is important to diagnose cancer with high sensitivity and specificity at the stage prior to treatment, a high cure rate can be achieved only when cancer is detected at an early stage through such diagnosis.

Thus, there is a need to develop a non-invasive, highly sensitive and highly specific method for diagnosing cancer at an early stage. However, until now, molecular diagnostic technology for diagnosing cancer by specifically detecting a lesion at an early stage is insignificant, and furthermore, there is no method specifically applied to specific cancer.

### PRIOR PATENT DOCUMENT

### Patent Document

Korea Patent Registration No. 10-2080887

### DETAILED DISCLOSURE OF THE INVENTION

### Technical Goals

Under this background, the present inventors continued research to develop a novel marker for diagnosing cancer or predicting prognosis derived from exosomes. As a result, the present disclosure has been completed by confirming that Tubulin alpha-1C chain (TUBA1C) protein specifically expressed in cancer cell-derived exosomes is used to diagnose cancer accurately and quickly or predict prognosis.

Accordingly, an aspect of the present disclosure is to provide a marker composition that may improve the accuracy of a cancer diagnosis while being used as a non-invasive method, including exosomes overexpressing Tubulin alpha-1C chain (TUBA1C) protein and a method for providing information necessary for diagnosing cancer or predicting prognosis using the same.

However, the goal to be achieved by the present disclosure is not limited to the above-mentioned goals, and other goals not mentioned will be clearly understood by to those of ordinary skill in the art from the following description.

### Technical Solutions

According to an example embodiment of the present disclosure, there is provided a marker composition for diagnosing cancer or predicting prognosis, including exosomes overexpressing Tubulin alpha-1C chain (TUBA1C) protein.

According to an aspect, the exosome may further include a GRIP and coiled-coil domain-containing protein (GCC2) protein.

According to an aspect, the cancer may be lung cancer, thymic cancer or esophageal cancer.

According to another example embodiment of the present disclosure, there is provided a composition for diagnosing cancer or predicting prognosis, including at least one of a primer or probe that specifically binds to the TUBA1C gene in the exosome; and an antibody that specifically binds to the TUBA1C protein in the exosome.

According to an aspect, the composition may further include at least one of a primer or probe that specifically binds to the GCC2 gene in the exosome; and an antibody that specifically binds to the GCC2 protein in the exosome.

According to an aspect, the cancer may be lung cancer, thymic cancer or esophageal cancer.

According to still another example embodiment of the present disclosure, there is provided a kit for diagnosing cancer or predicting prognosis, including the composition.

According to an aspect, the kit may be at least one selected from the group consisting of an RT-PCR kit, a microarray chip kit, a DNA kit, and a protein chip kit.

According to yet another example embodiment of the present disclosure, there is provided a method for providing information necessary for diagnosing cancer or predicting prognosis, including a step of measuring an expression level of the TUBA1C gene or protein in an exosome isolated from a biological sample.

According to an aspect, the method may further include a step of measuring an expression level of the GCC2 gene or protein in the exosome.

According to an aspect, the biological sample may be at least one selected from the group consisting of whole blood, serum, plasma, saliva, urine, sputum, lymph, and cells.

### Effects

The marker composition of the present disclosure may include the TUBA1C protein that is specifically and highly expressed in exosomes of cancer patients, to non-invasively and highly accurately diagnose cancer or predict the prognosis by measuring its expression level.

In addition, the marker composition of the present disclosure may further improve the sensitivity and accuracy of cancer diagnosis by using TUBA1C and GCC2 overexpressed in exosomes as dual biomarkers.

It should be understood that the effects of the present disclosure are not limited to the above-described effects and include all effects that can be inferred from the configuration of the invention described in the detailed description or claims of the present disclosure.

### BRIEF DESCRIPTION OF DRAWINGS

FIG. 1 is a diagram illustrating an ELISA result comparing the expression levels of GCC2 and TUBA1C proteins from the blood-derived exosomes of a lung cancer patient and a normal group.

FIG. 2 is a diagram illustrating an ELISA result comparing the expression level of TUBA1C protein in plasma-derived exosomes of esophageal cancer patients and a normal control group.

FIG. 3 is a diagram illustrating an ELISA result comparing the expression level of TUBA1C protein in plasma-derived exosomes of thymic cancer patients with and a normal control group.

FIG. 4 is a diagram illustrating an ELISA result measuring the expression levels of TUBA1C and GCC2 proteins in the blood-derived exosomes of lung cancer patients, which are divided by stage.

FIG. 5 is a diagram illustrating a ROC curve confirming a change in the sensitivity of diagnosis of lung cancer, compared to when TUBA1C and GCC2 are used alone, when the two markers are used in combination.

### BEST MODE FOR CARRYING OUT THE INVENTION

Hereinafter, example embodiments will be described in detail with reference to the accompanying drawings. The same reference numerals described in each drawing indicate the same elements.

Various modifications may be made to the example embodiments described below. It should be understood that the example embodiments described below are not intended to limit the example embodiment formation and include all modifications, equivalents, and substitutions thereto.

Terms used in the example embodiments are only used to describe specific example embodiments and are not intended to limit the example embodiments. The singular expression includes the plural expression unless the context clearly dictates otherwise. It is to be understood that in the present specification, terms such as “comprise” or “have” are intended to designate that a feature, number, step, operation, component, part, or combination thereof described in the specification exists, but it does not preclude the possibility of the presence or addition of one or more other features, numbers, steps, operations, components, parts, or combinations thereof.

Unless defined otherwise, all terms used herein, including technical or scientific terms, have the same meaning as commonly understood by one of ordinary skill in the art to which the example embodiment belongs. Terms such as those defined in a commonly used dictionary should be interpreted as having a meaning consistent with the meaning in the context of the related art and should not be interpreted in an ideal or excessively formal meaning unless explicitly defined in the present application.

Further, in the description with reference to the accompanying drawings, the same components are assigned the same reference numerals regardless of the reference numerals, and the overlapping description thereof will be excluded. In the description of the example embodiment, if it is determined that a detailed description of a related known technology may unnecessarily obscure the gist of the example embodiment, the detailed description thereof will be excluded.

According to an example embodiment of the present disclosure, there is provided a marker composition for diagnosing cancer or predicting prognosis, including exosomes overexpressing Tubulin alpha-1C chain (TUBA1C) protein. In addition, the exosome may further include a GRIP and coiled-coil domain-containing protein (GCC2) protein.

As used in the present specification, the terms “exosome overexpressing TUBA1C protein” and “exosome overexpressing GCC2 protein” refer to exosomes expressing GCC2 or TUBA1C protein at a higher level than exosomes present in normal cells.

An exosome is a small endoplasmic reticulum of nano-size (30 nm to 150 nm) secreted by most cells. It is known that various types of proteins, genetic materials (DNA, mRNA and miRNA), lipids, etc., derived from cells are contained in the exosome inside and phospholipid double membranes. Further, it has been reported that tissue-derived exosomes may be used for the diagnosis of diseases because they reflect the state of the tissue that secreted them.

Accordingly, the present inventors have completed the present disclosure by confirming that TUBA1C or GCC2 protein specifically expressed in exosomes of cancer patients may be used to accurately and quickly diagnose cancer or predict the prognosis.

Here, cancer includes all cancers, which, for example, include lung cancer, esophageal cancer, thymus cancer, breast cancer, liver cancer, stomach cancer, colorectal cancer, pancreatic cancer, cervical cancer, skin cancer, prostate cancer, ovarian cancer, thyroid cancer, bladder cancer, head and neck cancer, bone marrow cancer, biliary tract cancer, but is not limited thereto, and preferably lung cancer, esophageal cancer or thymus cancer.

As used in the present specification, the term “diagnosis” refers to determining the presence or characteristics of a pathological condition, i.e., whether or not cancer has developed. Further, “prognosis” refers to determining recurrence, metastases, drug reactivity, or resistance in the subject after cancer treatment. This may include the concept of predicting whether or not the subject has a good survival prognosis in the future, as well as whether the subject has cancer by measuring the expression level of TUBA1C or GCC2 in the exosomes isolated from the subject's sample.

As such, the expression level of TUBA1C or GCC2 protein derived from exosomes is measured to diagnose cancer or predict the prognosis, so a primer or probe that specifically binds to its gene, or an antibody that specifically binds to a protein may be used as a composition for diagnosing cancer or predicting prognosis.

Further, the present disclosure provides a kit for diagnosing cancer or predicting prognosis to which any one or more of a primer or a probe that specifically binds to a TUBA1C or GCC2 gene and an antibody that specifically binds to a TUBA1C or GCC2 protein are applied.

The kit may include an RT-PCR kit, a microarray chip kit, a DNA kit, a protein chip kit, and the like, but is not limited thereto. The kit may determine and detect the expression level of TUBA1C or GCC2 gene or protein corresponding to the marker in the exosome, thereby diagnosing lung cancer or predicting prognosis

The kit may include one or more other component compositions, solutions, or devices suitable for analysis methods, in addition to primers, probes, or antibodies that selectively recognize markers for diagnosing cancer or predicting prognosis.

For example, the kit may include a substrate, a suitable buffer solution, a secondary antibody labeled with a chromogenic enzyme or a fluorescent substance, a chromogenic substrate, and the like for the immunological detection of the antibody. Further, the substrate may include a nitrocellulose membrane, a 96-well plate synthesized from a polyvinyl resin, a 96-well plate synthesized from a polystyrene resin, a slide glass made of glass, etc., the chromogenic enzyme may include peroxidase, alkaline phosphatase, etc., the fluorescent substance may include FITC, RITC, etc., the chromogenic substrate solution may include 2,2′-azino-bis-(3-ethylbenzothiazoline-6-sulfonic acid) (ABTS), 0-phenylenediamine (OPD) and tetramethyl benzidine (TMB), but is not limited thereto.

According to another example embodiment of the present disclosure, there is provided a method of providing information necessary for diagnosing cancer or predicting prognosis, including a step of measuring the expression level of the TUBA1C gene or protein in the exosome isolated from the biological sample.

The method of the present disclosure may further include a step of measuring the expression level of the GCC2 gene or protein in the exosome in order to improve the sensitivity and accuracy for diagnosing cancer.

The biological sample may be one or more selected from the group consisting of whole blood, serum, plasma, saliva, urine, sputum, lymph and cells, and preferably whole blood or cells, but is not limited thereto.

The step of measuring gene expression level is a process of confirming the presence and expression level of mRNA of TUBA1C and GCC2 genes from a biological sample for diagnosing cancer or predicting prognosis, which means a step of measuring the mRNA expression level.

Analysis methods for this include reverse transcription polymerase reaction (RT-PCR), competitive reverse transcription polymerase reaction (competitive RT-PCR), real-time reverse transcription polymerase reaction (real-time RT-PCR), RNase protection assay (RPA), northern blotting, a DNA chip, etc, but is not limited thereto.

Further, the step of measuring protein expression level refers to a process of confirming the presence and expression level of TUBA1C and GCC2 proteins from a biological sample for diagnosing cancer or predicting prognosis.

The method of measuring protein expression level or comparative analysis includes protein chip analysis, immunoassay, ligand binding assay, matrix assisted laser desorption/ionization time of flight mass spectrometry (MALDI-TOF) analysis, surface enhanced laser desorption/ionization time of flight mass spectrometry (SELDI-TOF) analysis, radioimmunoassay, radioimmunodiffusion method, Ouchterlony immunodiffusion method, rocket immunoelectrophoresis, tissue immunostaining, complement fixation assay, two-dimensional electrophoresis analysis, liquid chromatography-mass spectrometry (LC-MS), liquid chromatography-mass spectrometry/mass spectrometry (LC-MS/MS), western blotting, enzyme-linked immunosorbent assay (ELISA), etc., but is not limited thereto.

After measuring the expression level of the TUBA1C, GCC2 gene or protein, when the expression level is higher than that of the normal control, it can be determined that cancer has occurred, or is highly likely to develop.

In addition, according to an example embodiment of the present disclosure, there is provided a method of screening cancer therapeutic agent, the method including steps of (a) processing a cancer drug candidate substance in a biological sample collected from a cancer patient; (b) isolating the exosomes from the biological sample; and (c) measuring the expression level of the TUBA1C gene or protein in the exosome.

As an extension of the method for providing information necessary for diagnosing cancer or predicting prognosis, the screening for candidate substances of therapeutic agents may be applied. That is, when after a biological sample isolated from a cancer patient is treated with a candidate substance of cancer therapeutic agents and the expression level of the TUBA1C gene or protein is decreased in the exosomes present therein, it can be confirmed that the candidate substance effectively functions as a cancer therapeutic agent.

Hereinafter, the present disclosure is described in more detail through Examples. The following Examples are described for the purpose of illustrating the present disclosure, but the scope of the present disclosure is not limited thereto.

### Example 1: Exosome Isolation and Proteomic Analysis Preparation

Five lung cancer cell lines (H522, A549, H1650, PC9, and H1299), thymic cancer cell lines, and esophageal cancer cell lines, respectively, were cultured in a 150 mm diameter dish. At this time, the supernatant of fetal bovine serum (FBS) from which exosomes were depleted by ultra-high speed centrifugation at 120,000 g for 4 hours was used as a culture medium. Using the culture medium, cells were continuously cultured for 2 to 3 days to reach 70% to 80% confluency.

The obtained culture medium was centrifuged at 10,000 g for 30 minutes to remove cell debris and passed sequentially through 0.45 μm and 0.22 μm filters to preferentially remove relatively bulky substances. Thereafter, the filtered cell culture medium was concentrated using an Amicon tube 100K (Millipore, USA), leaving only particles of the desired size.

Next, only the particles of the exosome size (50 nm to 100 nm) were separated from the concentrated cell culture medium using the column liquid chromatography method and concentrated again using an Amicon tube 100K.

Proteins were obtained from the concentrated exosomes using RIPA lysis buffer (Thermo Fisher Scientific, USA), and the result of the proteomic analysis was obtained by requesting the Korea Basic Science Institute (KBSI).

Based on this, Tubulin alpha-1C chain (TUBA1C) and GRIP and coiled-coil domain-containing protein 2 (GCC2), which are overexpressed in exosomes of cancer cell lines, were finally selected.

### Example 2: Measurement of GCC2 and TUBA1C Expression Levels in Exosomes of Lung Cancer Patients

In order to confirm whether the exosomes containing the GCC2 and TUBA1C proteins selected in Example 1 can be used as a marker for diagnosing lung cancer or predicting prognosis, expression levels of GCC2 and TUBA1C from exosomes extracted from the blood of the normal group (n=3) and the lung cancer patient group (n=5) were analyzed by enzyme linked immunoassay (ELISA).

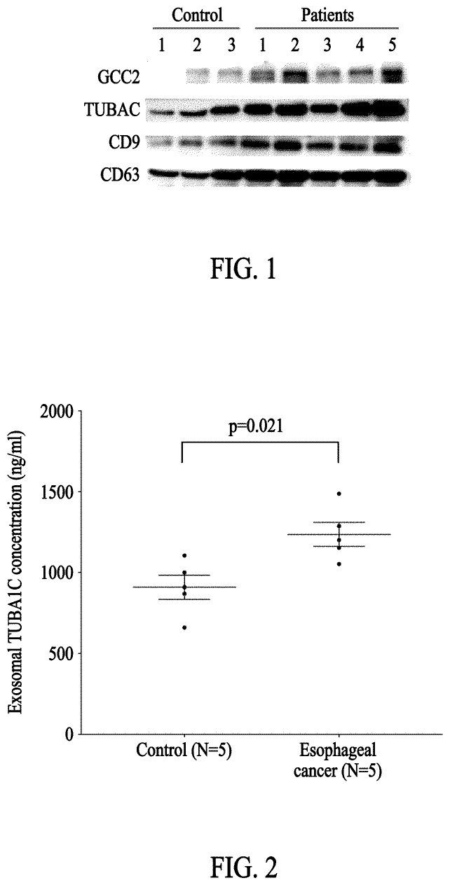

As shown in FIG. 1, the results indicated that the expressions of GCC2 and TUBA1C increased in the lung cancer patient group compared to the normal group (control).

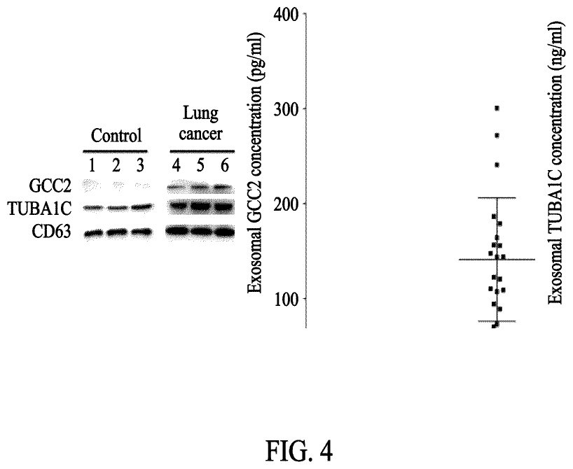

Next, in order to confirm the characteristics of exosomes isolated from the plasma of lung cancer patients, blood was collected from 20 patients with stage 1 to 3 lung cancer visiting the hospital, and exosomes were isolated from plasma using Exoquick (Systembio, USA). Expression levels of GCC2 and TUBA1C derived from isolated blood-derived exosomes were confirmed through ELISA analysis (GCC2: Mybiosource's GRIP and coiled-coil domain containing protein 2 ELISA KIT (Cat No. MBS9330667), 2) and TUBA1C: Mybiosource's TUBA1C ELISA KIT (Cat No. MBS9336377)). As shown in FIG. 4, the results indicated that the expression levels of GCC2 and TUBA1C significantly increased in all stages of lung cancer compared to the normal group, and the expression levels of GCC2 and TUBA1C also increased as the stage of lung cancer increased.

### Example 3: Measurement of TUBA1C Expression Level in Exosomes of Esophageal and Thymic Cancer Patients

First, exosomes were extracted from plasma samples of five normal subjects and five esophageal cancer patients, and then the TUBA1C protein concentration in the sample was confirmed using the TUBA1C ELISA KIT (Mybiosource's TUBA1C ELISA KIT (Cat No. MBS9336377).

As shown in FIG. 2, the results indicated that the average concentration of exosome-derived TUBA1C protein in normal subjects was 939.306 ng/ml, whereas, in esophageal cancer patients, the average concentration of exosome-derived TUBA1C protein was high as 1236.764 ng/ml. The concentration of exosome-derived TUBA1C protein in esophageal cancer patients was increased 1.36 times compared to normal subjects, and the p-value was 0.021, confirming that it was statistically significant.

Next, after extracting the exosomes from the plasma samples of 5 normal subjects and five thymic cancer patients, the TUBA1C protein concentration in the sample was confirmed using the TUBA1C ELISA KIT (Mybiosource's TUBA1C ELISA KIT (Cat No. MBS9336377).

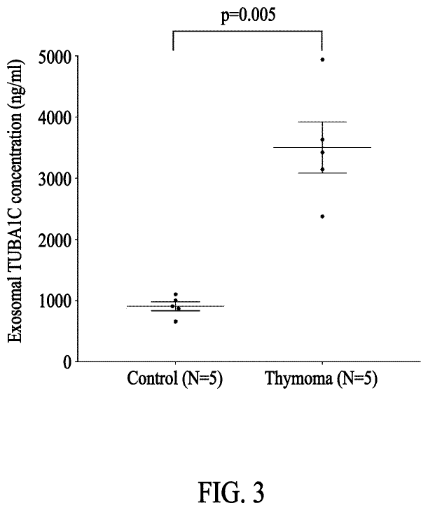

As shown in FIG. 3, the results indicated that the average concentration of exosome-derived TUBA1C protein in normal subjects was 909.306 ng/ml, whereas, in esophageal cancer patients, the average concentration of exosome-derived TUBA1C protein was very high as 3503.15 ng/ml. The concentration of exosome-derived TUBA1C protein in esophageal cancer patients was increased 3.85 times compared to normal subjects, and the p-value was 0.005, confirming that it was statistically significant.

### Example 4: Assessment of Diagnostic Utility as Dual Biomarkers of TUBA1C and GCC2

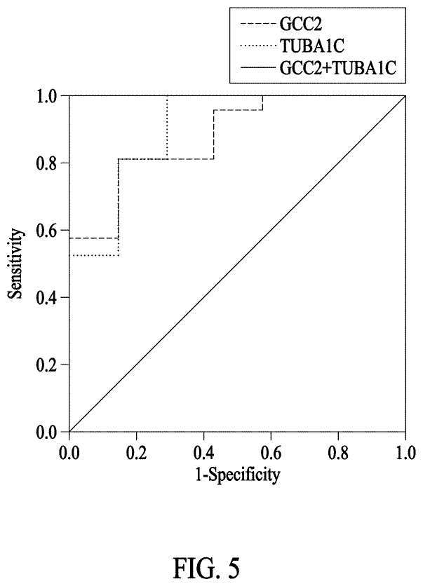

In order to evaluate the usefulness of using TUBA1C and GCC2 as dual biomarkers for diagnosing cancer, the changes in diagnostic sensitivity when TUBA1C and GCC2 were used alone and when both markers were used in combination were confirmed with a ROC curve. Specifically, after extracting exosomes from plasma from 7 normal subjects and 21 lung cancer patients, the concentrations of GCC2 and TUBA1C proteins, respectively, in the exosomes were obtained using GCC2 ELISA KIT and TUBA1C ELISA KIT, and then the AUC value was statistically confirmed using the ROC curve. The results are shown in FIG. 5.

Referring to FIG. 5, when the GCC2 antibody was used alone, the AUC was 0.905 (p=0.002), and when the TUBA1C antibody was used alone, the AUC value was 0.8787 (p=0.003). On the other hand, when GCC2 and TUBA1C antibodies were treated simultaneously, the AUC was 1 (P=0.0000963), indicating that TUBA1C and GCC2 are used as dual biomarkers to allow more precise diagnosis than when TUBA1C or GCC2 is used alone.

As described above, although the Examples have been described with reference to the limited Examples and drawings, various modifications and variations are possible from the above description by those skilled in the art. For example, even if the described techniques are performed in an order different from the described method, and/or the described components are combined or jointed in a different form from the described method or are replaced or substituted by other components or equivalents, appropriate results can be achieved.

Therefore, other implementations, other example embodiments, and equivalents to the claims are also within the scope of the following claims.

1. A marker composition for diagnosing cancer or predicting prognosis, the marker composition comprising exosomes overexpressing Tubulin alpha-1C chain (TUBA1C) protein.

2. The marker composition of claim 1, wherein the exosome further includes a GRIP and coiled-coil domain-containing protein (GCC2) protein.

3. The marker composition of claim 1 or 2, wherein the cancer is lung to cancer, thymic cancer or esophageal cancer.

4. A composition for diagnosing cancer or predicting prognosis, the composition comprising:
at least one of a primer or probe that specifically binds to TUBA1C gene in an exosome; and an antibody that specifically binds to TUBA1C protein in the exosome.

5. The composition of claim 4, further comprising:
at least one of a primer or probe that specifically binds to GCC2 gene in the exosome; and an antibody that specifically binds to GCC2 protein in the exosome.

6. The composition of claim 4 or 5, wherein the cancer is lung cancer, thymic cancer or esophageal cancer.

7. A kit for diagnosing cancer or predicting prognosis, the kit comprising the composition of claim 4 or 5.

8. The kit of claim 7, wherein the kit is at least one selected from the group consisting of an RT-PCR kit, a microarray chip kit, a DNA kit, and a protein chip kit.

9. A method for providing information necessary for diagnosing cancer or predicting prognosis, the method comprising a step of measuring an expression level of TUBA1C gene or protein in an exosome isolated from a biological sample.

10. The method of claim 9, further comprising:
a step of measuring an expression level of GCC2 gene or protein in the exosome.

11. The method of claim 9 or 10, wherein the biological sample is at least one selected from the group consisting of whole blood, serum, plasma, saliva, urine, sputum, lymph, and cells.
